(19) United States
(12) Patent Application Publication
(10) Pub. No.: US 2020/0038230 A1
(43) Pub. Date: Feb. 6, 2020

(54) Funnel and Tube for Urination

(71) Applicant: Faith H. Leibman, Narberth, PA (US)

(72) Inventor: Faith H. Leibman, Narberth, PA (US)

(21) Appl. No.: 16/600,386

(22) Filed: Oct. 11, 2019

Related U.S. Application Data
(63) Continuation-in-part of application No. 15/612,160, filed on Jun. 2, 2017.

(51) Int. Cl. A61F 5/455

(52) U.S. Cl. CPC A61F 5/4556; A61F 2005/4402

(57) ABSTRACT

A funnel has a collar surrounding a flow axis. A surface, defined by lobes surrounds and faces the flow axis. The surface has an extended lateral shape afforded by different sized lobes. A tube extends from the funnel. The tube is curved about a center of curvature.

### CROSS REFERENCE TO RELATED APPLICATIONS

This application is a continuation in part of and claims priority to application Ser. No. 15/612,160, filed Jun. 2, 2017, which is hereby incorporated by reference in its entirety.

### FIELD OF THE INVENTION

The invention concerns tubes extending from funnels, for example, for aiding with female urination.

### BACKGROUND

Men often urinate in a standing position specifically when camping, traveling, at festivals, on long car journeys, or any kind of outdoor pursuit where the toilet facilities are absent or filthy. Women face challenges distinct from men when urinating in a standing position because unlike men, they have less control over the direction of their urine stream. Of particular concern is the capture of lateral urine spray in the direction of the coronal body plane. There is clearly a need for a device which addresses the challenges faced by women in this regard.

### SUMMARY

The invention concerns a funnel having a tube extending therefrom. In one example embodiment the tube comprises an inlet end and an outlet end. The funnel comprises a collar surrounding a bore defining a coaxial flow axis. The collar is adapted to receive the inlet end of the tube. A plurality of lobes are attached to the collar. The lobes define a surface surrounding and facing the flow axis. A first and a second one of the lobes are positioned on one side of the flow axis. A third and a fourth one of the lobes are positioned on an opposite side of the flow axis. The span of the lobes, measured along a first line oriented perpendicular to the flow axis, is greater than the span of the lobes measured along a second line perpendicular to the first line and the flow axis.

As an example, each of the lobes has a curved perimeter. In an example, each of the lobes has a curvature extending circumferentially about the flow axis. As an example, each of the lobes has a curvature extending between a perimeter of each lobe and the collar.

In an example, the funnel further comprises a handle attached to at least one of the lobes. The handle is positioned along the greater span of the lobes. In a particular example, the handle comprises a tab oriented transversely to the flow axis and projecting away therefrom.

As an example, the tube is tapered. In a particular example, the tube comprises an outlet diameter and an inlet diameter, the outlet diameter being less than the inlet diameter.

As an example, the tube is curved about a center of curvature. In a particular example, the center of curvature is located in a plane defined by the first line and the flow axis. In another particular example, the center of curvature is located in a plane defined by the second line and the flow axis. In a particular example, the center of curvature is located in a plane defined by a third line and the flow axis. The third line is oriented transversely to the first and second lines.

In an example, the inlet end comprises first screw threads, and the collar comprises second screw threads. The second screw threads are compatible with the first screw threads. Engagement between the first and second screw threads attaches the inlet end to the collar. In a particular example, the first screw threads are male screw threads and the second screw threads are female. In another example, the first screw threads are female screw threads and the second screw threads are male.

As an example, the collar and the inlet end are attached in an interference fit. In a particular example, an outer surface of the inlet end engages a surface of the collar facing the flow axis. In another example, an outer surface of the collar engages an inner surface of the inlet end.

### BRIEF DESCRIPTION OF THE DRAWINGS

FIG. 1 is a top view of an example embodiment of a funnel and a tube according to the invention;

FIG. 2 is a sectional view taken at line 2-2 of FIG. 1 showing an example embodiment of the funnel and a portion of the tube;

FIG. 3 is an isometric view of an example embodiment of the combination funnel and the tube according to the invention;

FIG. 4 is a side view of an example embodiment of the tube;

FIG. 5 is a bottom view of an example embodiment of the funnel and the tube according to the invention showing an orientation of the center of curvature;

FIG. 6 is a bottom view of an example embodiment of the funnel and the tube according to the invention showing an orientation of the center of curvature; and

FIG. 7 is a bottom view of an example embodiment of the funnel and the tube according to the invention showing an orientation of the center of curvature.

### DETAILED DESCRIPTION

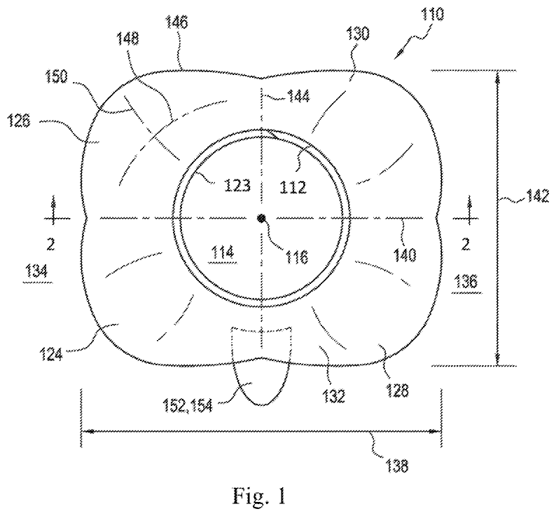

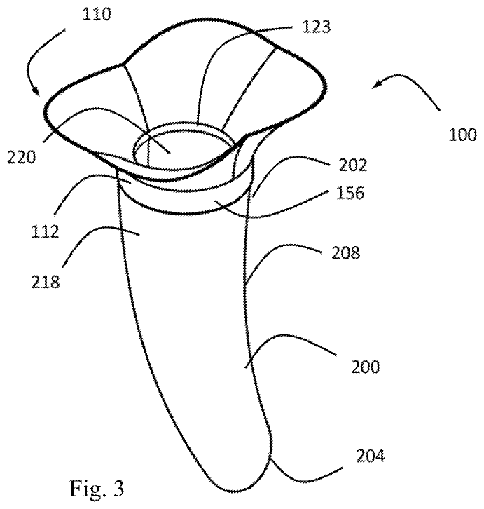

FIGS. 1, 2 and 3 show an example embodiment of a funnel 110 having a tube 200 extending therefrom, according to the invention. Funnel 110 comprises a collar 112 surrounding a bore 114 which defines a flow axis 116. Tube 200 comprises and inlet end 202 and an outlet end 204 (FIG. 3). Tube 200 also comprises an outer surface 218 and an inner surface 220.

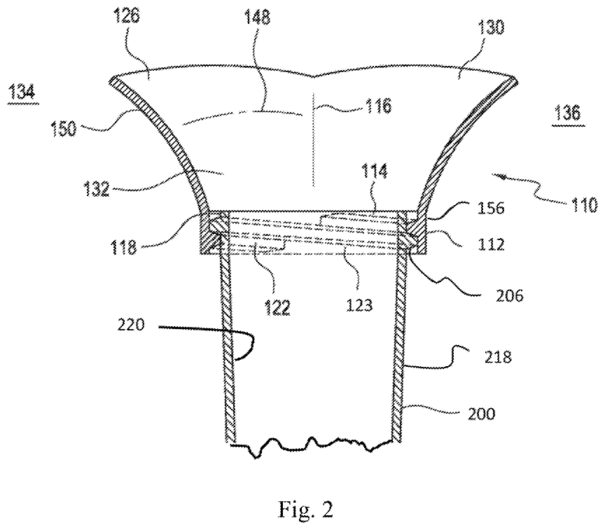

As shown in FIGS. 2 and 3, the collar 112 is adapted to receive the inlet end 202 of tube 200. The inlet end 202 comprises first screw threads 206 (see, FIG. 2). The collar 112 comprises second threads 122 which are compatible with the first screw threads 206. The first and second threads 206 and 122 engage and attach the inlet end 202 to the collar 112. The first screw threads 206 may be male screw threads and the second screw threads 122 may be female. Alternatively, the first screw threads 206 may be female screw threads and the second screw threads 122 may be male.

The collar 112 and the inlet end 202 may be attached in an interference fit, as shown in FIG. 3. The outer surface 218 of the inlet end may engage a surface 123 of the collar facing the flow axis 116 (FIG. 2). The outer surface 156 of the collar may engage the inner surface 220 of the inlet end. Alternatively, the inlet end 202 may be integral with the collar 112.

A plurality of lobes, in this example four lobes 124, 126, 128 and 130, are attached to the collar 112, as shown in FIGS. 1 and 2. The lobes define a surface 132 which surrounds and faces the flow axis 116. First and second lobes 124 and 126 are positioned on one side 134 of the flow axis 116, the third and fourth lobes 128 and 130 are positioned on the opposite side 136 of the flow axis. In this example embodiment, the span 138 of the lobes 124, 126, 128 and 130, measured along a first line 140 oriented perpendicular to the flow axis 116 is greater than the span of the lobes 142 measured along a second line 144 oriented perpendicular to both the first line 140 and the flow axis 116.

Each lobe 124, 126, 128 and 130 may have a curved perimeter 146, and each lobe may also have a compound curvature, i.e., each lobe may have a curvature 148 extending circumferentially about the flow axis 116, and each lobe may have a curvature 150 extending from a perimeter 146 of the lobe to the collar 112.

As shown in FIG. 1 a handle 152 may be attached to at least one of the lobes. Handle 152 is positioned along the side of the funnel 110 having the greater lobe span 138. In this example embodiment the handle 152 comprises a tab 154 oriented transversely to the flow axis 116 and projecting away therefrom.

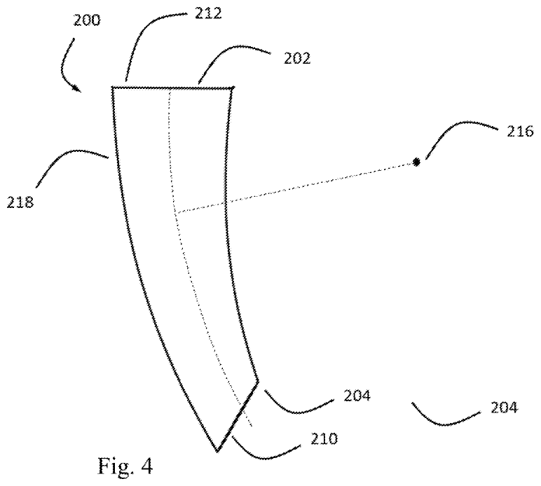

An example embodiment of tube 200 is shown in FIG. 4. The tube 200 may be tapered. The tube 200 comprises an outlet diameter 210 and an inlet diameter 212. The outlet diameter 210 may be less than the inlet diameter 212. Tube 200 is also curved about a center of curvature 216.

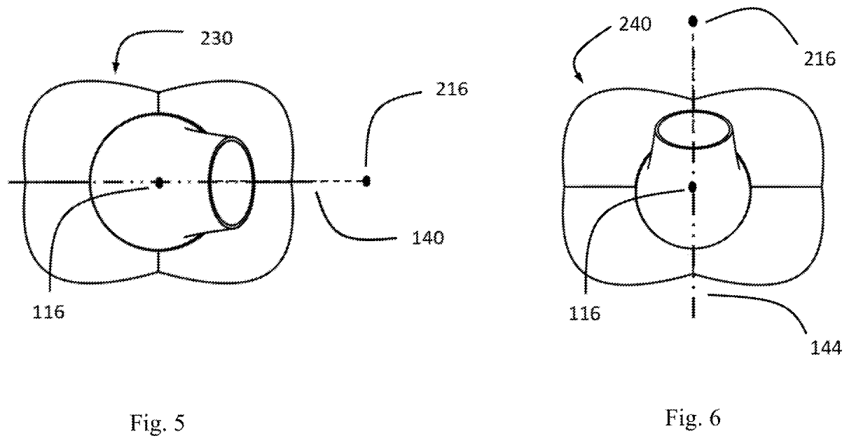

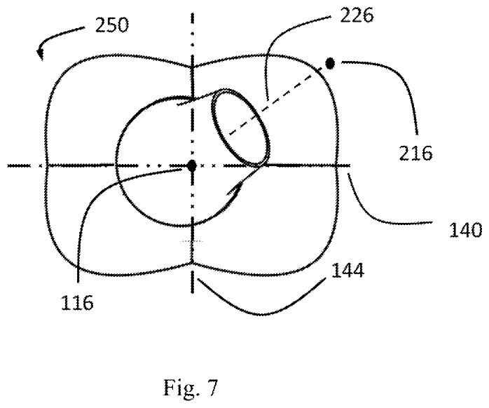

Example embodiments of the funnel with various orientations of the center of curvature 216 are shown in FIGS. 5, 6 and 7. FIG. 5 shows embodiment 230 where the center of curvature 216 may be located in a plane defined by the first line 140 and the flow axis 116. FIG. 6 shows embodiment 240 where the center of curvature 216 may be located in a plane defined by the second line 144 and the flow axis 116. FIG. 7 shows embodiment 250 where the center of curvature 216 may be located in a plane defined by a third line 226 and the flow axis 116. The third line 226 is oriented transverse to the first and second lines 140 and 144.

Example funnel 110 according to the invention is advantageously made of polymers such as polypropylene. Such materials permit an inexpensive and light weight design appropriate for one time use and disposal. In particular, when the funnels are made of polypropylene it permits them to be folded into a smaller size for economical packaging. Alternatively, funnel 110 may be reusable and may be made of metal, and other polymers that are not disposable.

The advantage of the lobed design of funnel 110 resides in its laterally extended shape which is expected to better capture lateral spray of a urine stream from the female urethra. In use, the handle 152 in funnel 110 is held between the thumb and index finger, the funnel is positioned between the legs of the user and the surface 123 is positioned beneath the urethra. When held in this manner the second and fourth lobes 126 and 130 face the posterior and the laterally extended shape of the funnel 110 afforded by the first, and third lobes 124, and 128 aligns appropriately to effectively capture lateral spray. Alternatively, in the absence of a handle, the surface opposite to surface 132 may be cupped by the fingers of the user to hold the funnel 110 in position.

What is claimed is:

1. A funnel having a tube extending therefrom, said tube comprising:
an inlet end and an outlet end;
said funnel comprising;
a collar surrounding a bore defining a flow axis, said collar adapted to receive said inlet end;
a plurality of lobes attached to said collar, said lobes defining a surface surrounding and facing said flow axis;
a first and a second one of said lobes being positioned on one side of said flow axis, a third and a fourth one of said lobes being positioned on an opposite side of said flow axis, the span of said lobes measured along a first line oriented perpendicular to said flow axis being greater than the span of said lobes measured along a second line perpendicular to said first line and said flow axis.

2. The funnel according to claim 1, wherein each of said lobes has a curved perimeter.

3. The funnel according to claim 1, wherein each of said lobes has a curvature extending circumferentially about said flow axis.

4. The funnel according to claim 1, wherein each of said lobes has a curvature extending between a perimeter of each said lobe and said collar.

5. The funnel according to claim 1, further comprising a handle attached to at least one of said lobes, said handle being positioned along said greater span of said lobes.

7. The funnel according to claim 5, wherein said handle comprises a tab oriented transversely to said flow axis and projecting away therefrom.

7. The funnel according to claim 1, wherein said tube is tapered.

8. The funnel according to claim 7, wherein said tube comprises an outlet diameter and an inlet diameter, said outlet diameter being less than said inlet diameter.

9. The funnel according to claim 1, wherein said tube is curved about a center of curvature.

10. The funnel according to claim 9, wherein said center of curvature is located in a plane defined by said first line and said flow axis.

11. The funnel according to claim 9, wherein said center of curvature is located in a plane defined by said second line and said flow axis.

12. The funnel according to claim 9, wherein said center of curvature is located in a plane defined by a third line and said flow axis, said third line being oriented transverse to said first and second lines.

13. The funnel according to claim 1, wherein said inlet end comprises first screw threads, said collar comprises second screw threads compatible with said first screw threads, engagement between said first and second screw threads attaching said inlet end to said collar.

14. The funnel according to claim 13, wherein said first screw threads are male screw threads and said second screw threads are female.

15. The funnel according to claim 13, wherein said first screw threads are female screw threads and said second screw threads are male.

16. The funnel according to claim 1, wherein said collar and said inlet end are attached in an interference fit.

17. The funnel according to claim 16, wherein an outer surface of said inlet end engages a surface of said collar facing said flow axis.

18. The funnel according to claim 16, wherein an outer surface of said collar engages an inner surface of said inlet end.
